(19) United States
(12) Patent Application Publication
(10) Pub. No.: US 2004/0186352 A1
(43) Pub. Date: Sep. 23, 2004

(54) Illumination system for medical diagnostic instrument

(75) Inventors: Chris R. Roberts, Skaneateles, NY (US); Corinn C. Fahrenkrug, Liverpool, NY (US); Allan I. Krauter, Syracuse, NY (US); Michael A. Pasik, Auburn, NY (US); John R. Strom, Moravia, NY (US); Peter J. Davis, Skaneateles, NY (US); Shawn A. Briggman, Syracuse, NY (US); Richard A. Tamburrino, Auburn, NY (US)

Correspondence Address: WALL MARJAMA & BILINSKI, 101 SOUTH SALINA STREET, SUITE 400, SYRACUSE, NY, 13202, US

(73) Assignee: Welch Allyn, Inc.

(21) Appl. No.: 10/393,340

(22) Filed: Mar. 20, 2003

(51) Int. Cl. A61B 1/267

(52) U.S. Cl. 600/200; 600/179

(57) ABSTRACT

A compact medical diagnostic instrument includes at least one white LED which is coupled to a light guide made up of a single optical fiber or a bundle containing a small number of optical fibers to provide illumination of a medical target, such as the ear canal.

### FIELD OF THE INVENTION

This invention relates to the field of medical diagnostic instruments, and more particularly to a low cost otoscopic assembly that utilizes at least one white LED and a light guide containing a single optical fiber or a bundle containing a small number of optical fibers to readily and efficiently direct light for illumination.

### BACKGROUND OF THE INVENTION

As is commonly known, an otoscope is a compact hand-held medical diagnostic instrument that is used to view the interior of the outer ear, and more specifically the tympanic membrane. An otoscopic instrument typically consists of a number of subassemblies arranged within an instrument housing including a battery subassembly, a power switch/regulation subassembly, a light source subassembly, a viewing lens, a light collection subassembly, and a light delivery subassembly. Most modern otoscopes utilize nickel-cadmium or alkaline batteries, as used with an incandescent lamp which comprises the light source subassembly. High performance otoscopes may use miniature halogen lamps for brighter light output. Power switches provide a graduated brightness through the use of a user-adjustable rheostat circuit, typically found on the exterior of the instrument handle. As to a light collection subassembly, many otoscopes use an integrated lens to collect the light from the light source and then focus the light into one end of a light guide consisting of a fiber optic bundle that is disposed in relation to a speculum portion which is inserted into the ear canal of a patient. The majority of the above features are described, for example, in U.S. Pat. No. 3,698,387 to Moore et al., as shown in FIG. 1.

Instruments, such as those described above, are inherently inefficient in their collection and delivery of light, therefore requiring inefficiently large light sources which drain their batteries in a relatively short period of time. In particular, the lamp energy is emitted omni-directionally from the lamp filament, with only a small portion of the total light emitted being collected for focusing purposes.

### SUMMARY OF THE INVENTION

It is a primary object of the present invention to overcome the above-noted deficiencies of the prior art.

It is another primary object of the present invention to provide a hand-held medical diagnostic instrument, such as an otoscope, ophthalmoscope or other similar device, which provides adequate illumination with at least similar or greater reliability and/or product life than those of currently known products.

It is yet a further object of the present invention to improve illumination uniformity, consistency of emitted illumination over the life of the battery, and battery life to those of currently known products.

Therefore and according to a preferred aspect of the invention, there is disclosed a medical diagnostic instrument for conducting at least one medical procedure, said instrument comprising:

a compact hand-holdable housing;

at least one white LED disposed within said housing for illuminating a medical target; and

a light guide coupled to said at least one white LED, said light guide comprising at least one of a single optical fiber and a bundle containing a small number of optical fibers having a light transmitting end disposed in a distal end of said housing for illuminating the medical target.

The light guide includes a light coupling end opposite the light transmitting end, with the light coupling end being coupled to the at least one white LED. This coupling can occur directly, such as through physical attachment of the light coupling end to the LED or LED die, or by providing at least one optical component, such as a lens, between the LED and the light coupling end of the light guide.

According to one embodiment of the invention, the optical component is part of an attachable member which can be releasably secured to the LED or to an LED housing. The light coupling end can also be made an integral part of the attachable member.

The light transmitting end of the light guide can preferably extend directly into the portion of the instrument housing which is used to view the medical target; for example, in the case of an otoscope, within the speculum.

Preferably, the instrument includes an eyepiece that permits visual examination along an optical path. Means are also preferably provided for masking the light of the contained white LED from the optical path ( i.e., such that this light cannot be seen by the viewer).

A single nine volt or other suitable battery can be used to operate the instrument, including the illumination system, the instrument further including circuitry means for regulating the power. As a result, little power is required to operate the instrument.

Preferably, the instrument is an otoscope used for examining the ear and the tympanic membrane. It should be realized, however, that other instruments, such as ophthalmoscopes, skin surface microscopes, and vaginoscopes, among others, can incorporate the inventive concepts described herein.

According to yet another preferred aspect of the present invention, there is provided an otoscope for examining the tympanic membrane, said otoscope comprising:

a compact hand-holdable housing;

at least one white LED disposed within said housing for illuminating the tympanic membrane;

a light guide coupled to said at least one white LED, said light guide comprising at least one of a single optical fiber and a bundle containing a small number of optical fibers, said light guide having a light transmitting end disposed in a distal end of said housing.

According to yet another preferred aspect of the present invention, there is provided a method for coupling a light guide to at least one white LED in a medical diagnostic instrument, said light guide comprising at least one of a single optical fiber and a bundle containing a small number of optical fibers, said method comprising the steps of:

boring a hole into the body of a white LED;

placing one end of a light transmissive optical fiber of said light guide into said hole and in proximity with the die and phosphor of said LED.

According to still another preferred aspect of the present invention, there is disclosed a method for coupling at least one white LED to a light guide, said light guide having at least one light conductive fiber, said method comprising the steps of:

placing at least one focusing lens element forward of said at least one white LED;

focusing light from said at least one LED to the end of at least one light conductive fiber of said light guide.

An advantage of the present invention is that the use of at least one white LED as a light source is more efficient, in that these light sources emit more light in the “forward” direction (that is, toward the intended target) than other known sources, such as bulbs, thereby providing nearly the same collectable light of a halogen bulb or lamp at a fraction of the power.

Another advantage achieved by the present invention is that less power being required allows the battery or other power source used in the diagnostic instrument to have a significantly longer life and in fact permits a much smaller power source to be utilized.

The outputted light is efficiently collected because the LED is at substantially lower (cooler) temperatures than conventional light sources, such as halogen lamps, therefore allowing plastic collection optics to be placed in substantially close proximity to the light source than is the case for conventional light sources.

A more efficient light delivery system can be provided than previously known illumination systems which rely upon bundles of glass fibers, that have large packing fraction losses (typically 25 percent or more).

In addition, LEDs in general have inherent advantages over incandescent light sources in that they are more rugged, are more reliable, have longer life, create less heat, consume less power, and have lower packaged cost.

These and other objects, features, and advantages will become readily apparent from the following Detailed Description which should be read in conjunction with the accompanying drawings.

### BRIEF DESCRIPTION OF THE DRAWINGS

FIG. 1 is a side elevational view of a hand-held medical diagnostic instrument made in accordance with the prior art;

FIG. 2 is a side elevational view, taken in section, of a hand-held medical diagnostic instrument made in accordance with a first preferred embodiment of the present invention;

FIG. 3 is a top perspective view of the tip of the medical diagnostic instrument of FIG. 2;

FIGS. 4 and 5 depict separate techniques for coupling the illumination output of a white LED with a single optical fiber;

FIG. 6 illustrates a schematic diagram of a voltage regulator circuit for use with the instrument of FIGS. 2-5;

FIG. 7 depicts a schematic diagram of another power regulation circuit;

FIG. 8 is a side view of a white LED which has been configured with a lens system for coupling thereto; and

FIG. 9 is a side view of a white LED configured with another light collecting optical system in accordance with a preferred embodiment according to the invention.

### DETAILED DESCRIPTION

The following description relates to certain versions of a hand-held otoscopic apparatus embodying the present invention used for examining the tympanic membrane of a patient. It should be readily apparent, however, that the inventive concepts described herein can similarly be incorporated into other medical diagnostic instruments, such as ophthalmoscopes, anoscopes, vaginoscopes, and the like. In addition, and throughout the course of discussion, certain terms are used which provide a frame of reference with respect to the accompanying drawings. These terms, however, unless otherwise stated, should not be regarded as limiting with regard to the inventive concepts described herein.

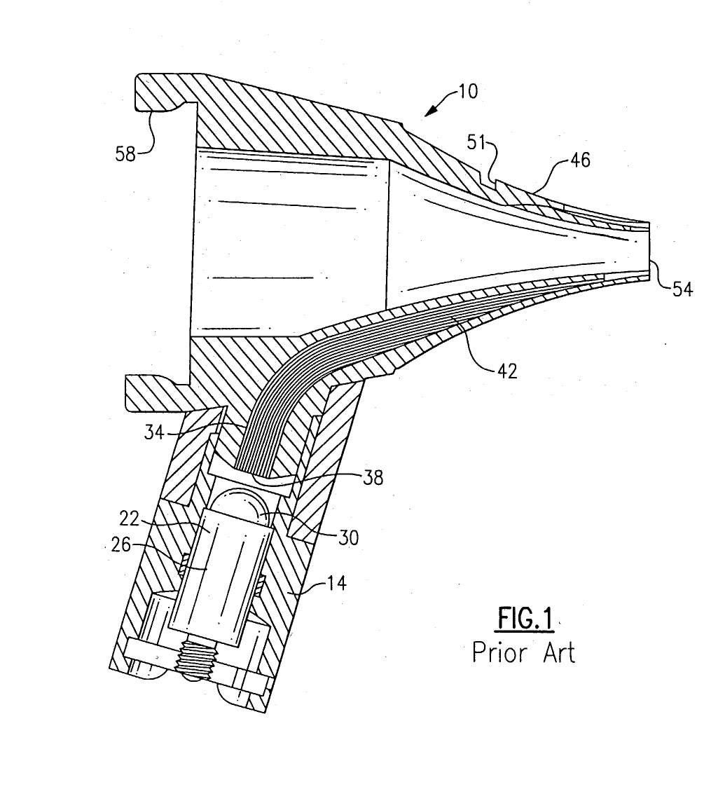

Referring to FIG. 1 and in order to provide sufficient background, a prior art otoscope is shown as 10. This known otoscope 10 includes a housing 14 that includes a power supply (not shown) in the form of batteries which are electrically connected to a lamp subassembly 22 which includes a lamp housing 26 defining a receptacle for an incandescent light source, such as a miniature halogen lamp 30. A light guide comprising a bundle of glass fibers 34 is provided having one end 38 positioned in relation to the lamp 30 and an opposite end 42 positioned within the circular tip opening 54 of a conically shaped insertion portion 46. The insertion portion 46 includes a distal end 50 onto which a speculum (not shown) is attached in a conventional manner to a bayonet 51. The speculum is sized to be fitted a predetermined distance into the ear canal of a patient (not shown). The bundle of optical fibers 34 of the light guide is preferably disposed about the circular tip opening 54 in a manner which does not prevent a user from visually inspecting the ear, via an aperture 58, through a magnifying eyepiece (not shown) provided on the proximal end of the top or head of the instrument. Additional details are found, for example, in U.S. Pat. No. 3,698,387 to Moore et al., the entire contents of which are herein incorporated by reference.

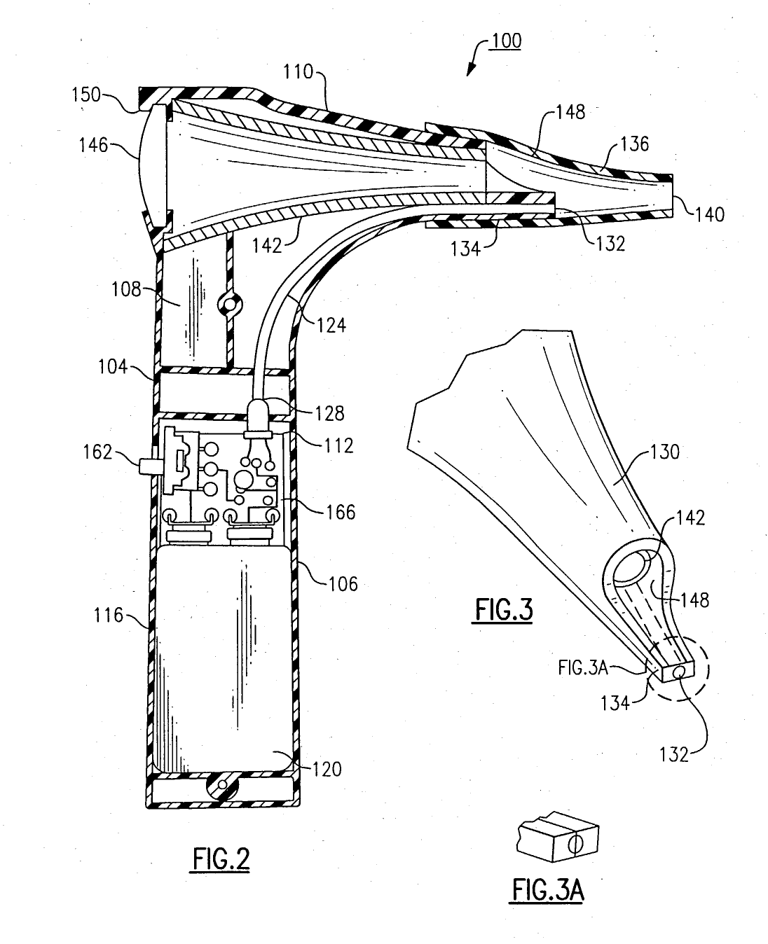

Referring to FIG. 2, there is shown a medical diagnostic instrument 100 in this instance, also an otoscope, made in accordance with a first embodiment of the invention. The instrument 100 includes a housing 104, like that described in FIG. 1, including a handle 106 and an instrument head 110 integrated with the top of the handle, which permits the entire instrument to be hand-held by a user. The housing 104 includes an interior 108 that contains a number of supported components which will now be described in greater detail.

According to the present invention and rather than using a halogen bulb or other incandescent light source, a single white LED 112, is placed in a lower part of the handle 106 adjacent a battery compartment 116 that supports at least one battery 120 such as a 9-volt, alkaline, nickel-cadmium or other suitable battery. The LED 112 is such as described in U.S. Pat. Nos. 5,998,925 and 6,069,440 to Shimuzu et al. and assigned to Nichia America, the entire contents of which are herein incorporated by reference. A light guide consisting of a single plastic optical fiber 124 is coupled at a first or light coupling end 128 directly to the white LED 112, the fiber having a second or light transmitting end 132 located within a user-attached speculum 136. Alternatively, the light guide can be comprised of a bundle containing a small number of optical fibers in lieu of the single plastic optical fiber 124.

A light mask or baffle 142 is disposed within an upper part of the housing interior 108 around a viewing lens 146 which is attached by conventional means at a proximal end 150 of the instrument head 110. The lens 146 serves as a magnifying eyepiece wherein an optical path is established between a distal end 130, FIG. 3, and the proximal end 150 of the instrument head 110 with the bottom of the light mask 142 forming a bottom wall thereof. This bottom wall isolates the optical path from the illumination output of the white LED 112. The bottom wall also isolates the optical path from most of the optical fiber 124. The optical path is isolated from the remainder of the optical fiber 124 of the light guide by locating the fiber 124 within the wall of the bottom extending portion 134 of the distal end 130 of the instrument 100, FIG. 3. The speculum 136 has a distal tip opening 140 and is attached in overlying relation onto the distal end 130 of the instrument head.

As shown more completely in FIG. 3, the bottom extending portion 134 of the distal end 130 of the instrument head 110 is extended distally relative to the viewing aperture 148 to permit the optical fiber 124 of the light guide to extend a greater distance within the speculum 136.

In lieu of a rheostat, an On/Off switch 162 is provided on the exterior of the handle 106 that is electrically connected to a circuit board 166 which interconnects the retained battery 120 and the single white LED 112.

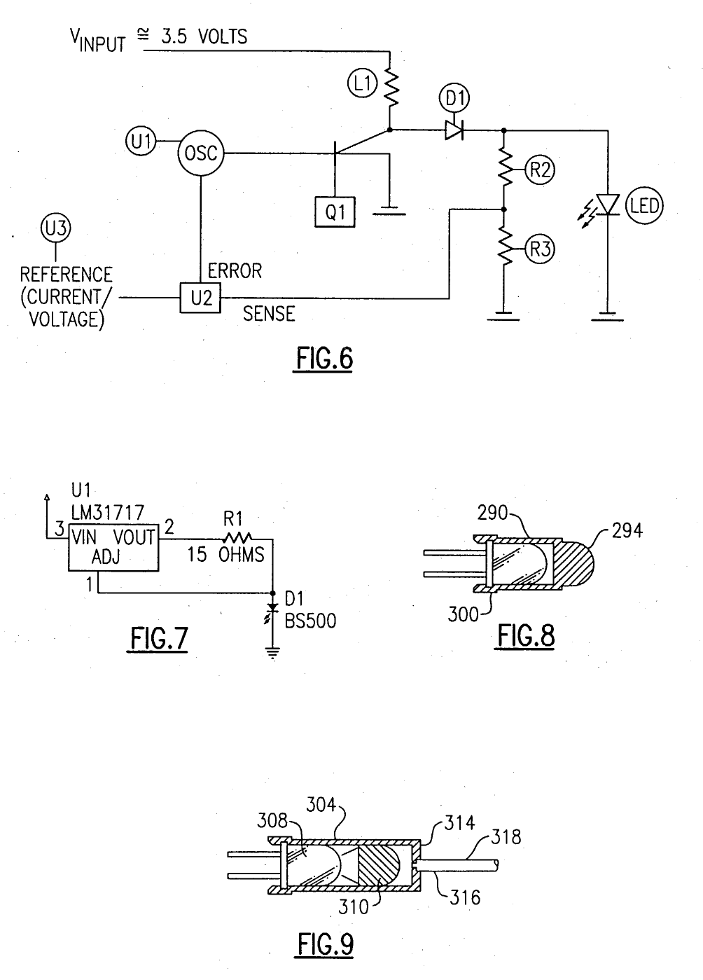

The On/Off switch 162 is attached to the circuit board 166. This board contains the electrical components required by the schematics of FIGS. 6 and 7 and also mounts the LED 112 and the contacts for the retained battery 120. The circuit provides a constant current to the LED 112 when the switch 162 is placed in the “On” position.

In operation, the instrument 100 is used by inserting the user-attached speculum 136 of the instrument head 110 into the ear canal of the patient (not shown). Activating the On/Off switch 162 energizes the LED 112, whose light is then conducted upwardly through the coupled end 128 of the optical fiber 124 to the light transmitting end 132 of the light guide located within the extending portion 134 that extends into the speculum 136 and provides illumination through the open tip opening 140. The user can view the target (e.g., the ear canal and tympanic membrane) along the optical path defined between the distal and proximal ends of the instrument head 110 while extraneous light from the white LED 112 is masked from the user by the baffle 142 and by the wall of extending portion 134.

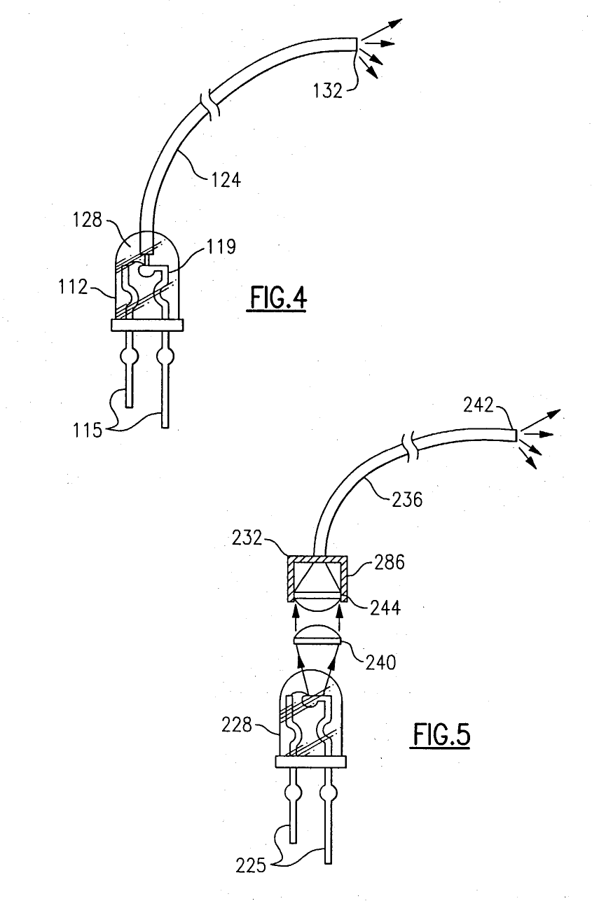

The technique of coupling the contained white LED to the plastic optical fiber will now be described in terms of certain embodiments, referring in turn to FIGS. 4, 5, 8 and 9. It will be readily apparent, however, that other variants are possible which are intended to be within the scope of the present invention.

Referring first to FIG. 4 and as shown in the otoscopic instrument of FIG. 2 above, the light coupling end 128 of the optical fiber 124 can be embedded directly into the body of a white LED 112 near the reflector cup, relative to the contained die and phosphor 119. In this instance, Nichia NSPW500BS or NSPW300BS white LEDs are used. A hole is drilled into the LED 112 and the end 128 of the light guide can be placed into the hole, which is then filled with an index-matching adhesive (e.g., a UV epoxy) or other suitable material in order to retain the sealed character of the LED 112. The coupled end 128 of the light guide is positioned relative to the die and phosphor 119 of the LED 112 prior to filling. In this instance, a preferred distance or gap of approximately 0.5 mm separates the light coupling end 128 and the die and phosphor 119.

Alternatively, and referring to FIGS. 5, 8 and 9 differing lens systems can be utilized to effectively couple light from the white LED to the end of an optical fiber of the light guide. As shown in FIG. 5, a pair of condenser lenses 240, 244, can be used and positioned distal of an LED 228 within the instrument. These lenses 240, 244, can be used to collect the exiting light from the LED 228 and collimate same and then focus the light into the coupling end 232 which can be attached to the distal most lens 244 by a holder 286.

In another embodiment, a LED housing 290 can be configured with an integral distal condenser lens element 294 as shown in FIG. 8, which receives the light from a contained white LED 300 and focuses same to a light coupling end (not shown) of a single optical fiber of a light guide. According to FIG. 9, and according to another embodiment, a LED housing 304 can retain an interior lens element 310 and further include a distal positioning portion 314 opposite a retained LED 308. The distal positioning portion 314 retains the light coupling end 316 of an optical fiber 318 of the light guide. It will be readily apparent that other suitable coupling techniques could be envisioned by one of sufficient skill in the field.

The power of the contained white LED needs to be regulated in order to allow it to be used. Typical power regulating circuits which can be used for the circuit boards of the instruments of FIGS. 2 and 4 are depicted in FIGS. 6 and 7.

For example and referring to FIG. 6, a circuit is illustrated which can be used to provide a constant current to the LED. As described in commonly owned and concurrently filed U.S. Ser. No. [to be assigned] [Attorney Docket 281_{—}378] and with relatively small modification, the circuit can be further configured to control the voltage of the LED or alternatively the color output of the LED by changing the sense feed line. The voltage control circuit functions as follows:

An oscillator (U1) is assumed to be a voltage controlled oscillator having a base frequency and its duty cycle that are a function of the input voltage. There are many PWM-type devices available, wherein this circuit does not rely on any particular such device. Upon initial power up of the circuit, the voltage across a pair of resistors (R2) and (R3) would be equal to zero, and the sense voltage would also be zero. Upon power-up of a comparator (U2), the sense voltage (zero at start) will be compared with the reference (U3) and a positive error signal will be generated. This error is fed to the oscillator (U1) which increases its on time cycle, thereby driving a transistor (Q1) to turn on. The preceding causes current to flow through inductor (L1) and stores energy as an electromagnetic field. During the “off” cycle of the oscillator (Q1) turns off and all current is then fed forward into diode (D1). This feeding creates a voltage to the LED which is sensed via a pair of voltage dividers (R2) and (R3). If this voltage remains lower than the reference (U3), the error comparator (U2) continues to generate a positive error and the oscillator continues to increase its pulse width which increases the energy which is stored in (L1), and consequently the output voltage into the LED. When the output and therefore the sense voltage becomes higher than the reference (U3), the error comparator (U2) generates a negative error signal which decreases the oscillator on time to reduce the output voltage. This process continues and maintains the output close to the reference voltage that is selected.

By including a resistor in series with the load rather than in parallel, the above circuit can easily control the current in the LED instead of the voltage. This arrangement is illustrated in FIG. 7. In addition, any other sensing means can be introduced such that the above circuit would respond to other changes such as light level, color, etc.

U1 is a constant voltage reference which maintains a constant voltage (Vref) between pin 1 and pin 2. A typical voltage for example, is approximately 1.2V. By placing a known resistor value between pin 1 and pin 2 (R1), the current through that resistor is controlled to a value equal to Vref/R1. As a consequence, any element that is put in series will also be controlled to this current value. In the present application, therefore placing the LED in series (connected to pin 2), will effectively control the current thereto.

Parts List for FIGS. 1-9

10 otoscope

14 housing

22 lamp subassembly

26 lamp housing

30 halogen lamp

34 optical fiber bundle

38 end

42 end

46 insertion portion

50 distal end

51 bayonet

54 tip opening

58 aperture

100 instrument

104 housing

106 handle

110 instrument head

112 white LED

115 contacts

116 battery compartment

119 die and phosphor

120 battery

124 optical fiber

128 light coupling end

130 distal end

132 light transmitting end

134 bottom extending portion

136 speculum

140 tip opening

142 light mask

146 viewing lens

148 aperture

150 proximal end

162 switch, on/off

225 LED electrical contacts

228 white LED

232 end

236 optical fiber

240 condenser lens

242 light transmitting end

244 condenser lens

260 conical shell member

264 proximal end

268 distal tip

272 switch, on/off

276 magnifier lens

280 proximal aperture

284 distal aperture

286 holder

290 LED housing

294 condenser lens element

300 LED

304 LED housing

308 LED

310 lens element

314 positioning portion

316 light coupling end

318 optical fiber

Though the invention has been described in terms of certain embodiments, it should be readily apparent that modifications and variations are possible which are within the intended scope of the invention as defined by the following claims.

We claim:

1. A medical diagnostic instrument for conducting at least one medical procedure, said instrument comprising:
a compact hand-holdable housing;
at least one white LED disposed within said housing for illuminating a medical target; and
a light guide coupled to said at least one white LED, said light guide comprising at least one of a single optical fiber and a bundle containing a small number of optical fibers, said light guide having a light transmitting end disposed in a distal end of said housing.

2. An instrument according to claim 1, wherein said light guide is made from plastic.

3. An instrument according to claim 1, wherein a first end of said light guide is directly coupled to said at least one white LED, said first end being opposite said light transmitting end.

4. An instrument according to claim 3, including at least one optical lens is disposed between said at least one white LED and said light coupling end of said light guide.

5. An instrument according to claim 1, including at least one battery power source contained in said housing for powering said at least one white LED.

6. An instrument according to claim 5, including circuit means for powering said at least one LED using said battery power source.

7. An instrument according to claim 5, including circuitry for powering said at least one LED with an effectively constant current using said battery power source.

8. An instrument according to claim 1, wherein said housing includes an eyepiece to permit viewing of the medical target, said instrument further including masking means for blocking light emanating from said at least one white LED from reaching said eyepiece.

9. An instrument according to claim 8, wherein said masking means further includes means for blocking light emanating from said light guide from reaching said eyepiece.

10. An instrument as recited in claim 1, wherein said instrument is an otoscope.

11. An instrument as recited in claim 1, wherein said instrument is an ophthalmoscope.

12. An otoscope for viewing the tympanic membrane, said otoscope comprising:
a compact hand-holdable housing;
at least one white LED disposed within said housing for illuminating the interior of the ear canal;
a light guide having a first end coupled to said at least one white LED, said light guide having a second light transmitting end disposed in a distal end of said housing wherein said light guide includes at least one of a single optical fiber and a bundle containing a small number of optical fibers.

13. An otoscope according to claim 12, wherein said light guide is made from plastic.

14. An otoscope according to claim 12, wherein said first end of said light guide is directly coupled to said at least one white LED.

15. An otoscope according to claim 12, including at least one optical lens is disposed between said at least one white LED and said light coupling end of said light guide.

16. An otoscope according to claim 12, including at least one battery power source contained in said housing for powering said at least one white LED.

17. An otoscope according to claim 16, including circuit means for powering said at least one LED using said battery power source.

18. An otoscope according to claim 16, including circuitry for powering said at least one LED with an effectively constant current using said battery power source.

19. An otoscope according to claim 12, wherein said housing includes an eyepiece to permit viewing of the ear canal, said otoscope further including masking means for blocking light emanating from said at least one white LED from reaching said eyepiece.

20. An otoscope according to claim 19, wherein said masking means further includes means for blocking light emanating from said light guide from reaching said eyepiece.

21. A method for coupling at least one white LED to a light guide, said light guide having at least one light conductive fiber, said method comprising the steps of:
boring a hole into the body of a white LED; and
placing one end of a light transmissive optical fiber of said light guide into said hole and in proximity with the die and phosphor of said LED.

22. A method for coupling at least one white LED to a light guide, said light guide having at least one light conductive fiber, said method comprising the steps of:
placing at least one focusing lens element forward of said white LED; and
focusing light from said LED to the end of at least one light conductive fiber of said light guide.

23. A method as recited in claim 22, wherein a said LED and at least one focusing lens element are integrated into a housing.

24. A method as recited in claim 22, wherein said at least one focusing lens element is made from plastic.

25. A method as recited in claim 22, wherein said at least one focusing lens element is integrally mounted on said at least one LED.

26. An instrument as recited in claim 3, wherein said light coupling end of said light guide is positioned within a hole bored into said at least one LED.

27. An instrument as recited in claim 4, wherein said at least one optical lens element and said at least one LED are retained within an integral housing.

28. An instrument as recited in claim 4, wherein said at least one optical lens element is made from plastic.

29. An instrument as recited in claim 4, wherein said at least one optical lens element is integrally mounted on said at least one LED.

30. An otoscope as recited in claim 14, wherein said light coupling end of said light guide is positioned within a hole bored into said at least one LED.

31. An otoscope as recited in claim 15, wherein said at least one optical lens and said at least one LED are retained within an integral housing.

32. An otoscope as recited in claim 15, wherein said at least one optical lens is made from plastic.

33. An otoscope as recited in claim 15, wherein said at least one optical lens is integrally mounted onto said at least one LED.
